(19) United States
(12) Patent Application Publication
(10) Pub. No.: US 2023/0084839 A1
(43) Pub. Date: Mar. 16, 2023

(54) ACTIVE AGENTS AGAINST CORONAVIRUS INFECTIONS AND DISEASES CAUSED THEREBY

(71) Applicant: Eberhard Karls Universität Tübigen Medizinische Fakultät, Tubingen (DE)

(72) Inventors: Michael Schindler, Reutlingen (DE); Natalia Ruetalo Buschinger, Tubingen (DE); Ramona Businger, Tubingen (DE)

(21) Appl. No.: 18/046,857

(22) Filed: Oct. 14, 2022

Related U.S. Application Data
(63) Continuation of application No. PCT/EP2021/059898, filed on Apr. 16, 2021.

(30) Foreign Application Priority Data
Apr. 17, 2020 (DE) 10 2020 110573.8

(51) Int. Cl. A61K 31/437; A61P 31/14

(52) U.S. Cl. CPC A61K 31/437; A61P 31/14

(57) ABSTRACT

The present invention relates to a novel agent for the prophylactic and therapeutic treatment of a coronavirus infection and/or a disease caused by said infection, a pharmaceutical composition containing said agent, and a method for the prophylactic and/or therapeutic treatment of a coronavirus infection and/or a disease caused by said infection.

### CROSS-REFERENCE TO RELATED APPLICATIONS

This application is a continuation of copending international patent application PCT/EP2021/059898 filed on 16 Apr. 2021 and designating the U.S.A., which has been published in German, and claims priority from German patent application DE 10 2020 110 573.8 filed on 17 Apr. 2020. The entire contents of these prior applications are incorporated herein by reference.

### FIELD OF THE INVENTION

The present invention relates to a novel active agent for the prophylactic and therapeutic treatment of a coronavirus infection and/or a disease caused by said infection, a pharmaceutical composition containing said active agent, and a method for the prophylactic and/or therapeutic treatment of a coronavirus infection and/or a disease caused by said infection.

The present invention relates to the field of molecular medicine, and more particularly to the field of antiviral agents.

### BACKGROUND OF THE INVENTION

The Coronaviridae, colloquially known as coronaviruses, are a family of viruses within the order Nidovirales. Their representatives cause very different diseases in various vertebrates such as mammals, birds and fish. The first coronaviruses were described in the mid-1960s. Coronaviruses are genetically highly variable. Individual species from the Coronaviridae family can also infect multiple species of hosts by overcoming the species barrier. Overcoming the species barrier has resulted in human infections with, among others, SARS-associated coronavirus (SARS-CoV, occasionally referred to as SARS-CoV-1)—the causative agent of the 2002/2003 SARS pandemic—and the Middle East respiratory syndrome coronavirus (MERS-CoV), which emerged in 2012.

The COVID-19 pandemic that originated in the Chinese city of Wuhan is attributed to a previously unknown coronavirus that was named SARS-CoV-2. The virus causes the viral disease COVID-19 (for corona virus disease 2019) and was the trigger of the COVID-19 pandemic, which was classified by the World Health Organization (WHO) as a “public health emergency of international concern” on 30 Jan. 2020, and as a pandemic on 11 Mar. 2020. The courses of the disease are nonspecific, diverse, and vary widely. In addition to asymptomatic infections, predominantly mild to moderate courses have been observed, but also severe ones with bilateral pneumonia up to lung failure and death.

### RELATED PRIOR ART

Currently, no specific treatment of COVID-19 is available, at best symptoms can be alleviated. Some existing virostatics are discussed, which are used for example against MERS-CoV and HIV. These include protease inhibitors such as indinavir, saquinavir, lopinavir/ritonavir, and interferon-beta, as well as the RNA polymerase inhibitor remdesivir.

As of 15 Feb. 2020, remdesivir, favipiravir, and chloroquine, an antimalarial agent, were in human trials in China. On 20 Mar. 2020, WHO launched the SOLIDARITY trial, which will evaluate remdesivir, chloroquine or hydroxychloroquine, lopinavir/ritonavir, and lopinavir/ritonavir with interferon-beta in thousands of patients worldwide.

The data obtained to date have not demonstrated convincing efficacy of the compounds tested. For this reason, there is an intensive search for new active agents against coronavirus infection, in particular one involving SARS-CoV-2 (COVID-19); see also Gordon et al. (2020), A SARS-CoV-2-Human Protein-Protein Interaction Map Reveals Drug Targets and Potential Drug-Repurposing; bioRxiv, preprint doi.org/10.1101/2020.03.22.002386. Promising candidates have not yet been found.

Against this background, the invention is based on the problem of providing an active ingredient that reduces, preferably avoids, the disadvantages from the prior art. In particular, an active ingredient is to be provided that can preferably counteract an infection with coronaviruses even in low concentrations and is thus suitable for the preparation of a medicament for the prophylaxis and/or treatment of a coronavirus infection and/or a disease caused by this infection.

The present invention satisfies these and other needs.

### SUMMARY OF THE INVENTION

This problem is solved by providing an inhibitor of bromodomain-containing protein (BRD) for the prophylaxis and/or treatment of a coronavirus infection and/or a disease caused by said infection.

According to the invention, “coronaviruses” include all species belonging to the Coronaviridae family of viruses, in particular SARS-CoV-2 (Sars-CoV-2, severe acute respira-tory syndrome coronavirus 2).

According to the invention, “treatment” of an infection and/or disease is understood in particular as a therapeutic intervention with the objective of positively influencing the overall health status of the treated living being determined by the infection and/or disease. In embodiments of the invention, the treatment comprises alleviating and/or reversing symptoms of the disease, reducing the viral load, eliminating the virus, and/or curing the disease.

According to the invention, “prophylaxis” of an infection and/or disease is understood in particular as a measure that serves to counteract an impairment of the overall state of health of the treated living being determined by the infection and/or disease. In embodiments of the invention, prophylaxis comprises prevention of a disease.

“Bromodomain” is a protein domain of approximately 110 amino acids that recognizes acetylated lysine residues, such as those at the N-terminal tails of histones. Bromodomains, as “readers” of lysine acetylation, are responsible for transducing the signal carried by acetylated lysine residues and converting it into various normal or abnormal phenotypes. “Bromodomain-containing proteins” (BRDs) can exert a variety of biological functions ranging from histone acetyltransferase activity and chromatin remodeling to transcription mediation and co-activation. Of the 43 BRDs known in 2015, 11 had two bromodomains and one protein had 6 bromodomains. The so-called BET family (bromodomain and extraterminal domain), a subgroup of the bromodomain family, includes, for example, the members BRD2, BRD3, BRD4, and BRDT. Preparation, biochemical analysis, and structure determination of the bromodomain-containing proteins have been described in detail in Ren et al. (2016), Preparation, Biochemical Analysis, and Structure Determination of the Bromodomain, an Acetyl-Lysine Binding Domain, Methods in Enzymology 573: 321-43. According to the invention, all bromodomain-containing proteins are covered.

According to the invention, an “inhibitor” of BRD, which is also synonymously referred to as a BRD inhibitor, is understood to be an agent that leads to selective and specific inhibition of BRD. BRD inhibitors are described, for example, in Bechter and Schoffski (2020), Make your best BET the emerging role of BET inhibitor treatment in malignant tumors, Pharmacology & Therapeutics 208:107479, but there under the synonymous designation BET inhibitors exclusively with respect to their anti-tumor activity.

According to the invention, inhibition may be by decreasing activity, functionality, expression, or other phenomena that result in inhibition of BRD. The agent may be in any conceivable material form, such as a small molecule, a peptide, an antibody, a nucleic acid molecule, etc.

The problem underlying the invention is hereby completely solved.

Using two model compounds, the inventors were surprisingly able to show experimentally that BRD inhibitors not only prevent the cytopathic effect caused by the virus but also reduce the viral load and the number of virus-infected cells. These effects are evident even at very low concentrations, which are in the nanomolar range. According to one embodiment of the invention, therefore, the BRD inhibitor is used at a concentration that is in the range of ≤1 μM, preferably ≤0.5 μM, further preferably ≤0.4 μM, further preferably 0.3 μM, further preferably 0.2 μM, at the cellular level (e.g., in human cells of the lung epithelium).

According to one embodiment of the invention, the infection and disease is an infection with a SARS-associated coronavirus (SARS-CoV) and a disease caused by this infection, preferably a SARS-CoV-2 infection and/or COVID-19.

With this measure, an active substance is provided in an advantageous manner which, according to the knowledge of the inventors, is superior to the substances currently being tested and also already clinically tested in part. Thus, the currently urgent need for active substances to combat COVID-19 is met in an advantageous manner.

According to a further embodiment of the invention, the inhibitor is an inhibitor of bromodomain-containing protein 2 (BRD2).

In a still further embodiment, the inhibitor is an inhibitor of bromodomaincontaining protein 4 (BRD4).

These measures have the advantage of inhibiting target structures that are known by the inventors to be of particular importance for the interaction of the virus with the infected host and thus for pathogenicity. Thus, a particularly effective inhibitor is provided in an advantageous manner. According to the invention, a BRD2 inhibitor and a BRD4 inhibitor can be two substances. Alternatively, it may be a single substance that inhibits both target structures and is referred to, for example, as a BRD2/4 inhibitor.

In humans, the “BRD4” protein is encoded by the BRD4 gene. It has two bomodomains, designated BD1 and BD2. It is homologous to the mouse MCAP protein, which associates with chromosomes during mitosis, and homologous to the human BRD2 (RING3) protein, a serine/threonine kinase. Each of these proteins contains two bromodomains, a conserved sequence motif that may be involved in chromatin targeting. BRD4 is frequently required for the expression of Myc and other “tumor-driving” oncogenes in hematologic cancers, including multiple myeloma, acute myeloid leukemia, and acute lymphoblastic leukemia.

In humans, the “BRD2” protein is encoded by the BRD2 gene. Early descriptions indicated that the BRD2 gene product is a mitogen-activated kinase located in the nucleus. The gene maps to the class II region of the major histocompatibility complex (MHC) on chromosome 6p21.3, but sequence comparison suggests that the protein is not involved in the immune response. Homology to the female sterile homeotic of the Drosophila gene suggests that this human gene may be part of a signal transduction pathway involved in growth control. BRD2 has been implicated in carcinogenesis and forms of obesity.

It has been found to be particularly advantageous in the invention that BRD2 and/or BRD4 inhibitors still show efficacy even at a particularly low concentration in the nanomolar range, whereas no effect at all is observed with active agents such as chloroquine. The inventors were able to demonstrate this exemplarily for two low molecular weight compounds in a cell culture experiment.

According to a further embodiment of the invention, the inhibitor is a lowmolecular compound.

According to the invention, a small molecule compound is a class of substances with a low molecular mass. They form the opposite group to larger, high-molecular substances, e.g., long-chain polymers. It includes active ingredients whose molecular mass does not exceed about 800 g mol⁻¹. Due to their small size, low molecular weight compounds are partially able to penetrate cells and exert their effects there.

According to another embodiment of the invention, the inhibitor is GSK2820151 (IBET151).

This measure has the advantage of providing an inhibitor that is particularly effective according to the findings of the inventors. It is already in clinical trials as an anti-tumor agent (Trail #NCT02630251) and is therefore rapidly available. It is currently manufactured by GlaxoSmithKline. It is described in Gadgeel et al. (2017), Abstract CT104: A first-in-human phase I dose escalation study of BET inhibitor GSK2820151 in patients with advanced or recurrent solid tumors cancers, Cancer Research 77, CT104, and in Bechter and Schöffski (op. cit.).

According to a still further embodiment of the invention, the inhibitor is 806870810/TEN-010 (CPI203).

This measure also has the advantage of providing an inhibitor that is particularly effective according to the knowledge of the inventors. It is already in clinical trials as an anti-tumor agent (trail #NCT01987362) and is therefore rapidly available. It is currently manufactured by Hoffmann-La Roche. It is described in Shapiro et al. (2015), Abstract A49: Clinically efficacy of the BET bromodomain inhibitor TEN-010 in an openlabel substudy with patients with documented NUT-midline carcinoma (NMC), Molecular Cancer Therapeutics 14 (12 Supplement 2), A49, and in Bechter and Schoffski (op. cit.).

Another subject-matter of the present invention relates to a pharmaceutical composition for the prophylaxis and/or treatment of a coronavirus infection and/or a disease caused by said infection, comprising the inhibitor according to the invention and a pharmaceutically acceptable carrier.

The features, properties, advantages and embodiments of the inhibitor according to the invention apply equally to the pharmaceutical composition according to the invention.

Pharmaceutically acceptable carriers are sufficiently known in the prior art. They include, for example, binders, disintegrants, fillers, lubricants, as well as buffers, salts and other substances suitable for the formulation of pharmaceuticals; see Rowe et al. (2012), Handbook of Pharmaceutical Excipients, 7th Edition Pharmaceutical Press; or Bauer et al. (2017), Lehrbuch der pharmazeutischen Technologie, 10th Edition, Wissenschaftliche Verlagsgesellschaft Stuttgart mbH. The contents of these publications are incorporated by reference into this application.

According to one embodiment of the pharmaceutical composition according to the invention, the inhibitor is present per dosage unit in a concentration which, after administration into a living being, results in a concentration which counteracts the coronavirus infection and/or a disease caused by this infection.

This measure has the advantage that the pharmaceutical composition contains the optimum amount of active ingredient necessary to achieve a therapeutic and/or prophylactic effect. For the precise adjustment of this concentration, a large number of test systems are available to the person skilled in the art, by means of which, depending on the living being to be treated, in particular the age, weight, any previous illnesses, the desired concentration can be determined individually.

According to one embodiment of the pharmaceutical composition according to the invention, the inhibitor is present per dosage unit in a concentration which, after administration into a living being, leads to a concentration in the coronavirus-infected cells, preferably the lung epithelial cells, which is about ≤10 μM, preferably about ≤1 μM, further preferably about ≤500 nM, further preferably about ≤400 nM, further preferably about 300 nM.

This measure advantageously takes into account the knowledge of the inventors that the BRD inhibitor already shows prophylactic or therapeutic effect in a coronavirus infection at very low concentrations. Side effects can thus be reduced or even avoided. Also, this measure contributes to the cost-effective production of the pharmaceutical composition.

According to a further embodiment of the pharmaceutical composition according to the invention, the latter comprises, in addition to the inhibitor according to the invention, a further active ingredient, preferably a further antiviral active ingredient.

Although the inhibitor according to the invention can be present as the sole active ingredient, this measure has the advantage that additive and possibly synergistic effects can lead to an enhancement of the prophylactic and therapeutic success.

Another subject-matter of the present invention relates to a method for the prophylactic and/or therapeutic treatment of a coronavirus infection and/or a disease caused by this infection, characterized by the administration of the inhibitor according to the invention and/or the pharmaceutical composition according to the invention to a living being, preferably a human being.

The features, properties, advantages and embodiments of the inhibitor according to the invention and the pharmaceutical composition according to the invention apply equally to the process according to the invention.

It is understood that the above-mentioned features and those to be explained below are usable not only in the combination indicated in each case, but also in other combinations or on their own, without leaving the scope of the present invention.

The present invention will now be explained in more detail with reference to examples of embodiments from which further features, characteristics and advantages of the invention are apparent. In this regard, the embodiment examples are not restrictive.

It is also understood that individual features disclosed in the embodiment examples are disclosed not only in the context of the specific embodiment in question, but in a generality, and in themselves make their own contribution to the invention. The person skilled in the art may therefore freely combine these features with other features of the invention.

In the embodiments, reference is made to the accompanying figures.

### BRIEF DESCRIPTION OF THE DRAWINGS

FIG. 1: Bright field micrographs of Calu3 cells left untreated (mock), infected with SARS-CoV-2 (SARS-CoV-2) and treated with 10 μM and 1 μM of the BRD2/4 inhibitor BET151 in addition to SARS-CoV-2 infection.

FIG. 2: Bright field micrographs of Calu3 cells left untreated (mock), infected with SARS-CoV-2 (SARS-CoV-2) and treated with 10 μM and 1 μM of the BRD2/4 inhibitor CPI203 in addition to SARS-CoV-2 infection.

FIG. 3: Bright field micrographs of Calu3 cells left untreated (mock), infected with SARS-CoV-2 (SARS-CoV-2) and treated with 10 μM and 1 μM of the CDK inhibitor RCB in addition to SARS-CoV-2 infection.

FIG. 4: Bright field micrographs of Calu3 cells left untreated (mock), infected with SARS-CoV-2 (SARS-CoV-2) and treated with 10 μM and 1 μM of the CDK inhibitor ACB in addition to SARS-CoV-2 infection.

FIG. 5: Bright field micrographs of Calu3 cells left untreated (mock), infected with SARS-CoV-2 (SARS-CoV-2) and treated with 10 μM and 1 μM of hydroxychloroquine (HChl) in addition to SARS-CoV-2 infection.

FIG. 6: Bright field micrographs of Calu3 cells left untreated (mock), infected with SARS-CoV-2 (inf) and treated with 10 μM, 5 μM and 2.5 μM of IBET151, CPI203 and HChl in addition to SARS-CoV-2 infection.

FIG. 7: Bright field micrographs of Calu3 cells left untreated (mock), infected with SARS-CoV-2 (inf) and treated with 1.25 μM, 0.612 μM and 0.306 μM of IBET151, CPI203 and HChl in addition to SARS-CoV-2 infection.

FIG. 8 Effects of BRD2/4 inhibitors on SARS-CoV-2 infection. Calu3 cells were infected with SARS-CoV-2 and treated with IBET151, CPI203, or hydroxychloroquin (HChl). At 24 hpi (hours post-infection), cells were fixed with 80% acetone and stained with DAPI and immunofluorescence against SARS-CoV-2. The number of cells was analyzed using a Cytation3 multiplicity imager. (A) Representative images, (B) quantification of total cell count (DAPI+), infected cells (IF+) and infection rate (IF+/DAPI+ cells), (C) Western blot analysis of Calu3 cell lysates using serum from a convalescent COVID-19 patient to detect SARS-CoV-2 proteins.

FIG. 9 Interaction of BRD2/4 inhibitors with SARS-CoV-2 cellular signaling pathways. Calu3 cells were infected with SARS-CoV-2 and treated with 2.5 μM IBET151 and CPI203. 24 hpi cells were lysed and pellets were subjected to Digi-West analyses (n=2, mock; n=6, inf.; n=2, IBET151; n=1, CPI203).

FIG. 10: Graph illustrating the effect of BRD2/4 inhibitors on the total number or survival of Calu3 cells after SARS-CoV-2 infection.

### DESCRIPTION OF PREFERRED EMBODIMENT

### 1. Material

### Test Substances

BRD2/4 inhibitor GSK2820151 (IBET151), GlaxoSmithKline
BRD2/4 inhibitor R06870810/TEN-010 (CPI203), Hoffmann-La Roche
CDK inhibitor Ribociclib (RCB)
CDK inhibitor abemaciclib (ACB)

### Hydroxychloroquine (HChl, HCQN)

### 2. Methodology

### Establishment of a SARS-CoV-2 Patient Isolate.

˜200 μl throat swab from a SARS-CoV2 positive patient was made up with 1 ml DMEM, sterile filtered (0.22 μM) and cultured for Caco2 cells. 2 days later, removal of supernatant, cell harvest and lysate generated for Western blot. Detection of SARS-CoV-2 proteins by western blot over serum from a COVID-19 convalescent. Confirmation SARS-CoV-2 via qRT-PCR analysis of the supernatant.

Residual supernatant cultured on new Caco2 cells for 2-5 days. Continuous harvest of supernatant as viral stock. Aliquoting and storage at −80° C.

### Testing of Substances for Antiviral Activity

Seed Calu3 (50,000 cells/well) in 96-well plate. 24 h later change medium to 170 μl DMEM+5% FCS (infection medium) per well. Prepare inhibitor dilutions so that after addition of 20 μl dilution the desired final concentration is reached. Then add 10-5 μl of virus stock (1:20, M01:0.4-1:40, M01:0.2) to the total volume of 200 μl. Incubate for another 24-48 h. After each 24 and 48 h, acquisition of transmitted light images via automated microscopy (4× magnification) using multiplate reader. Evaluation of the cytopathic effect of the virus infection via visual assessment of the cell layer.

TABLE 1
Data row 1
Data row 1:
Calu3 50.000 cells/well Inf Sep. 4, 2020
we seed the cells in 170 μl, 1 day later add 10 μl of virus
stock (Tü1) and 20 μl or pre-diluted compounds to each well

| | Nix | IBET151 | CPL203 | HChl | IBET151 | CPL203 | HChl | Nix | |
|---|---|---|---|---|---|---|---|---|---|
| PBS | PBS | PBS | PBS | PBS | PBS | PBS | PBS | PBS | PBS |
| PBS | Mock | 10 | 10 | 10 | 10 | 10 | 10 | Mock | PBS |
| PBS | Mock | 5 | 5 | 5 | 5 | 5 | 5 | Mock | PBS |
| PBS | Mock | 2.5 | 2.5 | 2.5 | 2.5 | 2.5 | 2.5 | Mock | PBS |
| PBS | inf | 1.25 | 1.25 | 1.25 | 1.25 | 1.25 | 1.25 | inf | PBS |
| PBS | inf | 0.625 | 0.625 | 0.625 | 0.625 | 0.625 | 0.625 | inf | PBS |
| PBS | inf | 0.3125 | 0.3125 | 0.3125 | 0.3125 | 0.3125 | 0.3125 | inf | PBS |
| PBS | PBS | PBS | PBS | PBS | PBS | PBS | PBS | PBS | PBS |

monitor the cells, at 1 dpi or potential 2 dpi harvest SN (in 96-Well plate). From the cells prepare images and lysates for cell titer glo (or MTT or SRB) store the SNs.

TABLE 2
Data row 2
Data row 2:
Calu3 50.000 cells/well Inf Sep. 4, 2020
we seed the cells in 170 μl, 1 day later add 10 μl of virus
stock (Tü1) and 20 μl or pre-diluted compounds to each well

| | Nix | IBET151 | CPL203 | HChl | IBET151 | CPL203 | HChl | Nix | |
|---|---|---|---|---|---|---|---|---|---|
| PBS | PBS | PBS | PBS | PBS | PBS | PBS | PBS | PBS | PBS |
| PBS | Mock | 10 | 10 | 10 | 10 | 10 | 10 | Mock | PBS |
| PBS | Mock | 5 | 5 | 5 | 5 | 5 | 5 | Mock | PBS |
| PBS | Mock | 2.5 | 2.5 | 2.5 | 2.5 | 2.5 | 2.5 | Mock | PBS |
| PBS | inf | 1.25 | 1.25 | 1.25 | 1.25 | 1.25 | 1.25 | inf | PBS |
| PBS | inf | 0.625 | 0.625 | 0.625 | 0.625 | 0.625 | 0.625 | inf | PBS |
| PBS | inf | 0.3125 | 0.3125 | 0.3125 | 0.3125 | 0.3125 | 0.3125 | inf | PBS |
| PBS | PBS | PBS | PBS | PBS | PBS | PBS | PBS | PBS | PBS |

monitor the cells, at 1 dpi or potential 2 dpi harvest SN (in 96-Well plate). From the cells prepare images and lysates for cell titer glo (or MTT or SRB) store the SNs.

### 3. Results

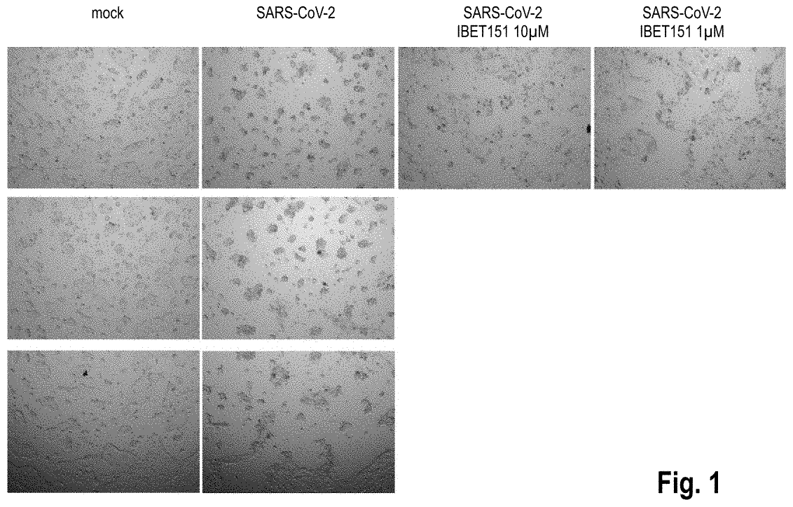

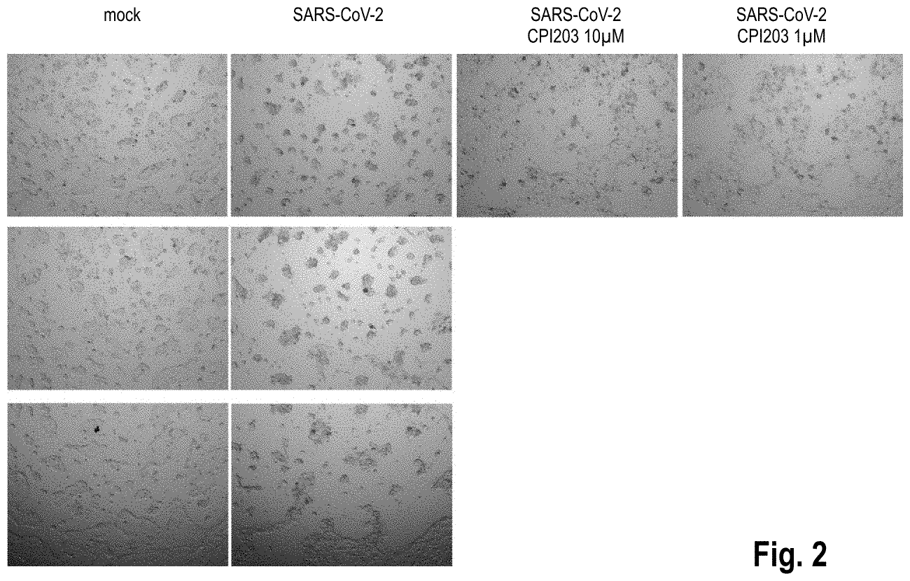

In the figures, the control approach is shown under “mock”. The used lung epithelial cells Calu3 show a flat structure in cell culture under the microscope. After infection of the cells with SARS-CoV-2 (images headlined with “SARS-CoV-2” or “inf”), there is a clear cytopathic effect, which is visible in the morphological change of the cell formations in the form of “clumps”, see e.g. FIG. 1-5, second column.

### Data Series 1:

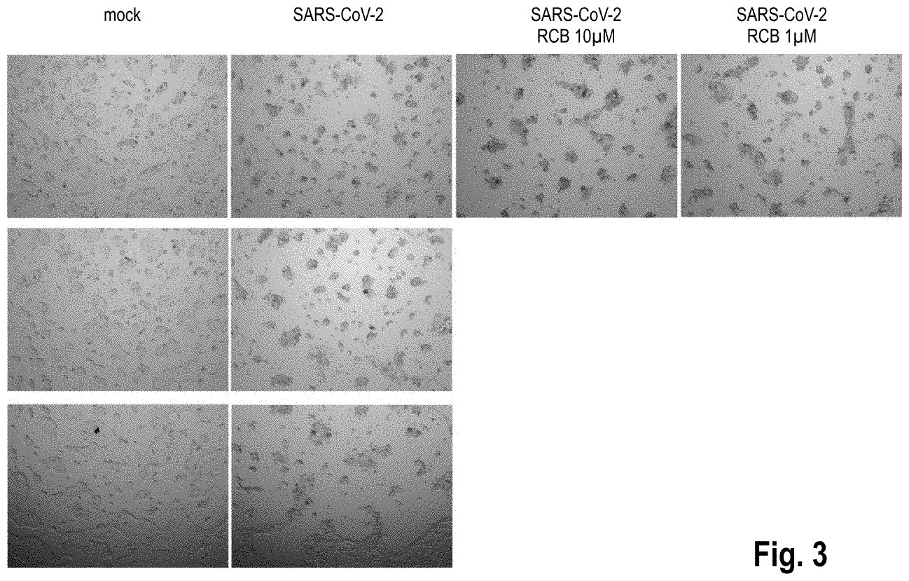

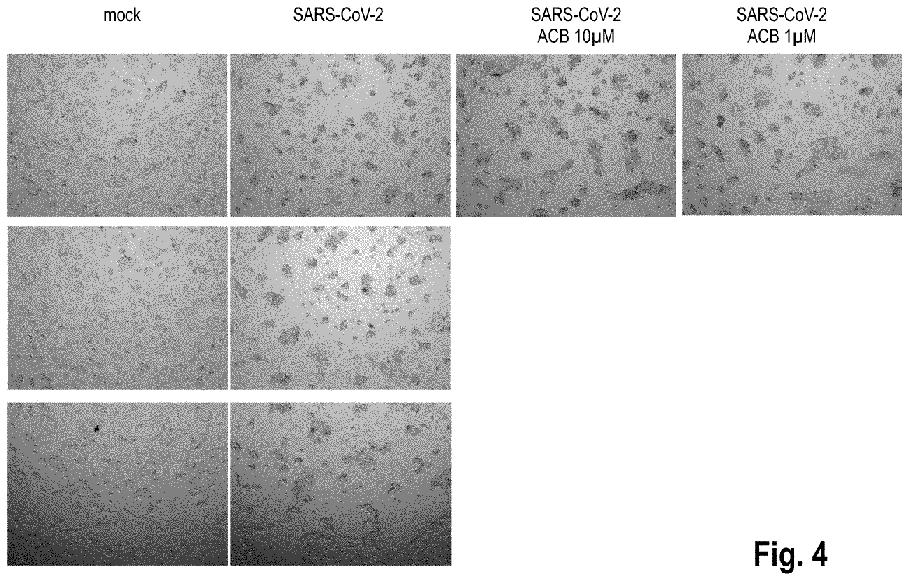

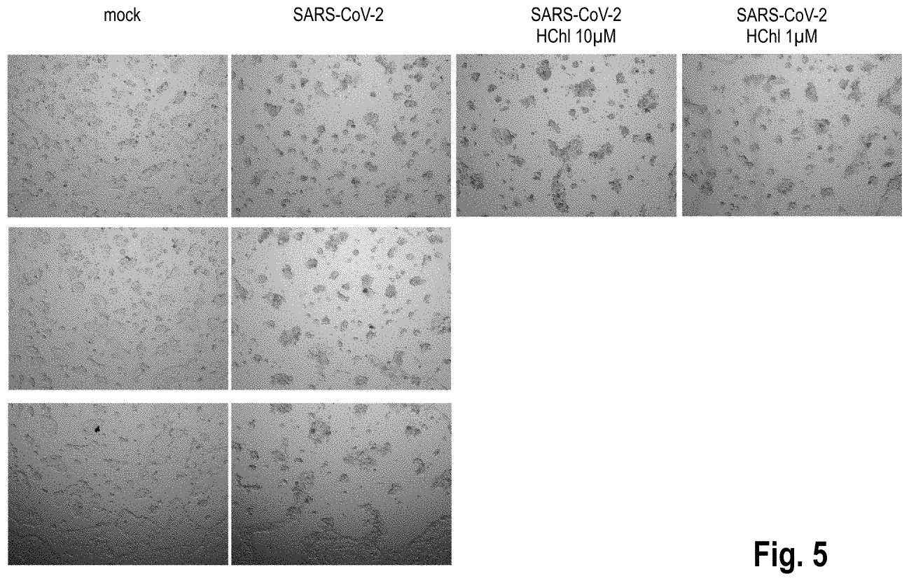

The BRD2/4 inhibitors IBET151 and CPI203 show a significant reduction of the cytopathic effect caused by viral infection at concentrations of 10 and 1 μM, respectively (comparison to mock/non-infected and SARS-CoV-2 infected); see FIGS. 1 and 2, 3rd and 4th columns. In contrast, equal concentrations of hyrdoxychloroquine (HChl, FIG. 5), as well as the clinically approved CDK inhibitors ribociclib (RCB; FIG. 3) and abemaciclib (ACB; FIG. 4), show no inhibitory effects.

### Data Series 2:

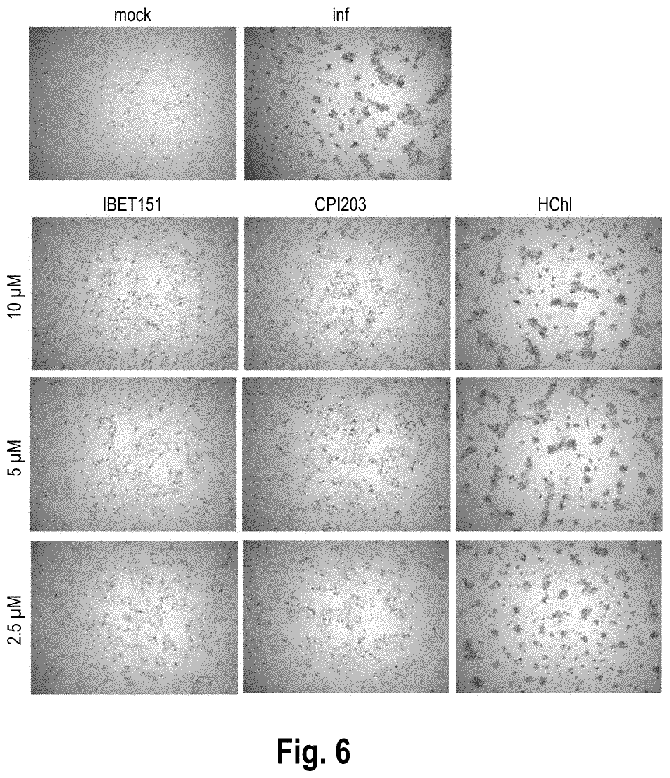

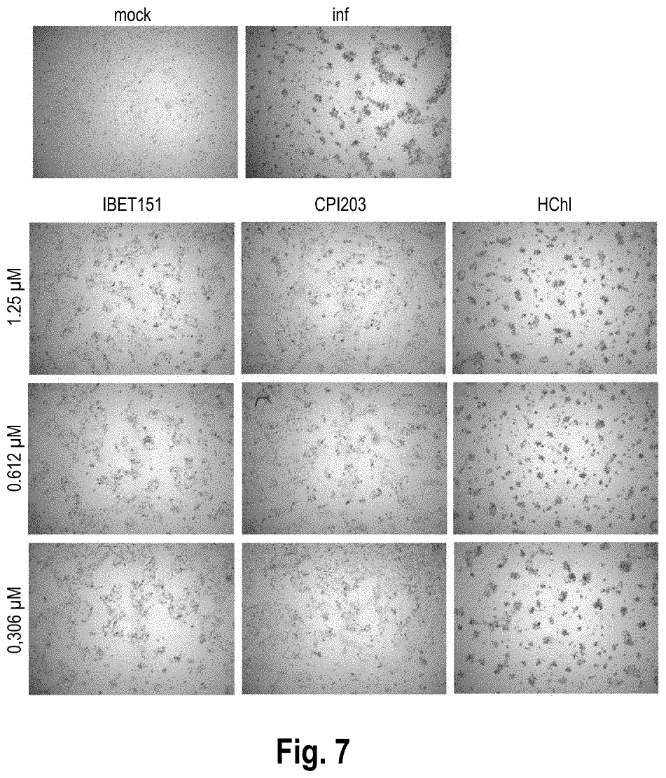

At a dose-dependent reduction in the concentrations of IBET151 and CPI203 of 10 μM-0.3125 μM, inhibitory effects on the virus-induced cytopathic effect are still evident even at the lowest concentrations, unlike at the same concentration of hydroxychloroquine (FIGS. 6 and 7).

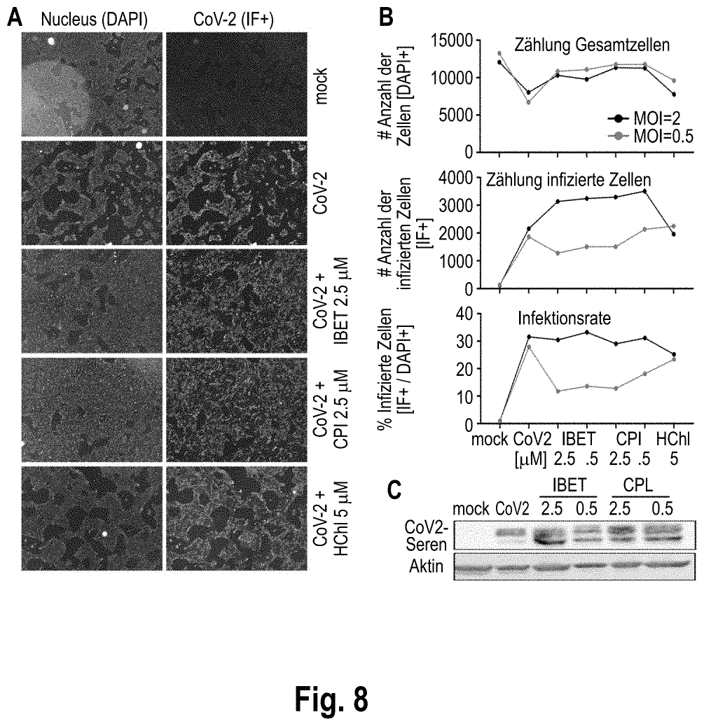

In further experiments, the inventors demonstrated that BRD2/4 inhibitor treatment of Calu3 cells infected with a high multiplicity of infection (MOI=2) of SARSCoV-2 completely blocked virus-induced cell death but did not affect virus production (FIG. 8). When high MOIs were used, the inventors did not observe a reduction in infected cells or total viral protein levels, although the virus-induced cytopathic effects were completely blocked. However, when infected with lower MOIs, the infection rate was reduced (FIG. 8B). This suggests that BRD proteins do not affect viral entry and infection or viral protein production but may influence the late steps of viral replication. This assumption is consistent with a possible role of E protein in coronavirus assembly and release.

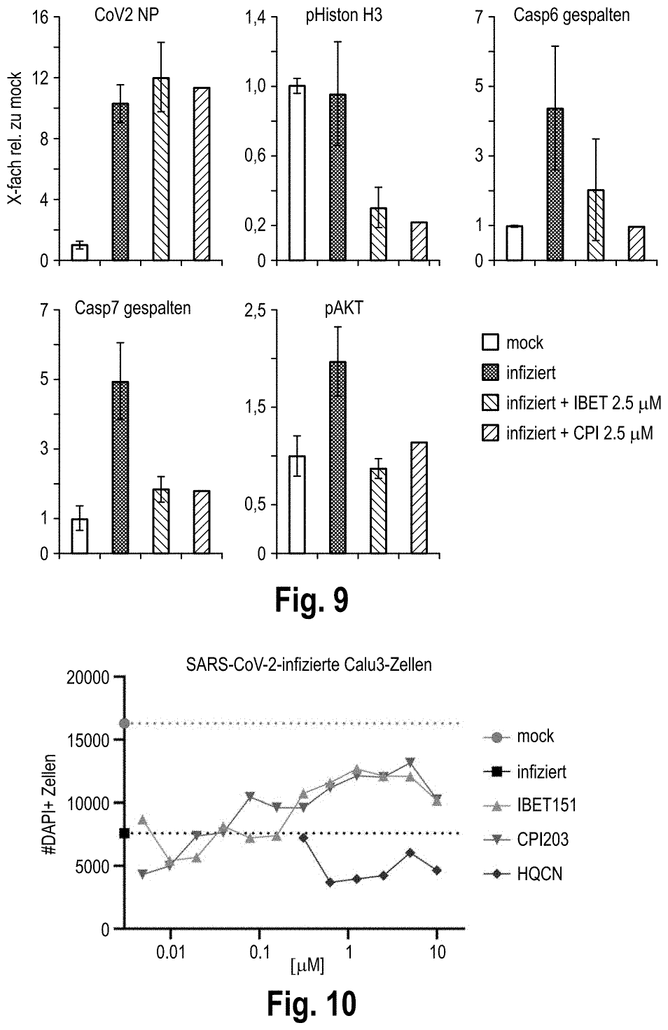

In further experiments, the inventors investigated the extent to which BRD2/4 inhibitors interfere with cellular signaling pathways used by SARS-CoV-2 to induce cytopathic effects. The inventors have included several controls that again confirm that when high MOIs are used for infection, the BRD2/4 inhibitors do not reduce viral protein production, as evidenced by the amount of viral nucleocapsid produced. As observed, BRD2/4 inhibitors block phosphorylation of histone H3, independent of SARS-CoV-2 infection. Remarkably, SARS-CoV-2 infection appears to induce an apoptotic signaling cascade, as evidenced by the induction of caspase 6/7 cleavage and AKT phosphorylation. All of these events are specifically blocked by BRD2/4 inhibitors Inhibitors, which may explain the lack of SARS-CoV-2-induced cell death when treated with the compounds (FIG. 9).

In FIG. 10, the effect of BRD2/4 inhibitors on the total number or survival of Calu3 cells after SARS-CoV-2 infection is again summarily illustrated. Calu-3 cells were infected or sham-infected (mock) with the clinical SARS-CoV-2 isolate. Simultaneously, cells were treated with both BRD2/4 inhibitors IBET151 and CPI203 and hydroxychloroquine (HCQN) as the reference drug at the indicated concentrations. At 48 hours post-infection, cells were fixed with 80% acetone and nuclei were counterstained with DAPI solution (2 mg/ml) for 10 minutes at RT. For quantification, the total amount of cells (DAPI+) was analyzed by automated microscopy. The average number of mock and infected cells is indicated with a dotted line. It can be seen that in the experiments performed, positive effects on the total number or survival of Calu3 cells after SARS-CoV-2 infection occur from a concentration of BRD2/4 inhibitors of approximately 20 nM. This is not observed in HQCN-treated cells. This observation was confirmed by the inventors in further experiments (not shown).

### 4. Conclusion

Using two selected example compounds, the inventors were able to demonstrate that inhibitors of bromodomain-containing protein (BRD) provide effective prophylactic and therapeutic treatment of coronavirus infection and/or disease caused by this infection, and thus represent a promising pharmacological option, particularly with respect to SARS-CoV-2 infection (COVID-19).

Therefore, What is claimed, is:

1. A method for the prophylactic or therapeutic treatment of a coronavirus infection or a disease caused by said infection, comprising the administration to a living being of an inhibitor of bromodomain-containing protein (BRD).

2. The method according to claim 1, wherein said infection is an infection with a SARS-associated coronavirus (SARS-CoV) and the disease is a disease caused by said infection.

3. The method according to claim 1, wherein said infection is an infection with SARSCoV-2 and wherein said disease is COVID-19.

4. The method according to claim 1, wherein said inhibitor is an inhibitor of bromodomain-containing protein 2 (BRD2).

5. The method according to claim 1, wherein said inhibitor is an inhibitor of bromodomain-containing protein 4 (BRD4).

6. The method according to claim 1, wherein said inhibitor is a low molecular weight compound.

7. The method according to claim 1, wherein said inhibitor is GSK2820151 (IBET151).

8. The method according to claim 1, wherein said inhibitor is R06870810/TEN-010 (CPI203).

9. The method according to claim 1, wherein the living being is a human being.

10. A pharmaceutical composition for the prophylaxis or treatment of a coronavirus infection or a disease caused by said infection, comprising an inhibitor of bromodomain-containing protein (BRD) and a pharmaceutically acceptable carrier.

11. The pharmaceutical composition according to claim 10, wherein said infection is an infection with a SARS-associated coronavirus (SARS-CoV) and the disease is a disease caused by said infection.

12. The pharmaceutical composition according to claim 10, wherein said infection is an infection with SARS-CoV-2 and wherein said disease is COVID-19.

13. The pharmaceutical composition according to claim 10, wherein said inhibitor is an inhibitor of bromodomain-containing protein 2 (BRD2).

14. The pharmaceutical composition according to claim 10, wherein said inhibitor is an inhibitor of bromodomain-containing protein 4 (BRD4).

15. The pharmaceutical composition according to claim 10, wherein said inhibitor is a low molecular weight compound.

16. The pharmaceutical composition according to claim 10, wherein said inhibitor is GSK2820151 (IBET151).

17. The pharmaceutical composition according to claim 10, wherein said inhibitor is 806870810/TEN-010 (CPI203).

18. The pharmaceutical composition according to claim 10, wherein the inhibitor is present per dosage unit in a concentration which, after administration into a living being, results in a concentration which inhibits coronavirus infection, or a disease caused by said infection.

19. The pharmaceutical composition according to claim 10, wherein the inhibitor is present per dosage unit at a concentration which, after administration into a living being, results in a concentration in the coronavirus-infected cells which is about ≤10 μM.

20. The pharmaceutical composition according to claim 10, wherein the inhibitor is present per dosage unit at a concentration which, after administration into a living being, results in a concentration in the coronavirus-infected cells which is about ≤1 μM.

21. The pharmaceutical composition according to claim 10, wherein the inhibitor is present per dosage unit at a concentration which, after administration into a living being, results in a concentration in the coronavirus-infected cells which is about ≤500 nM.

22. The pharmaceutical composition according to claim 10, wherein the inhibitor is present per dosage unit at a concentration which, after administration into a living being, results in a concentration in the coronavirus-infected cells which is about ≤400 nM.

23. The pharmaceutical composition according to claim 10, wherein the coronavirus-infected cells are lung epithelial cells.

24. The pharmaceutical composition according to claim 10, wherein it comprises a further active ingredient.

25. The pharmaceutical composition according to claim 24, wherein said further active ingredient is a further anti-viral active ingredient.
